(19) United States
(12) Patent Application Publication
(10) Pub. No.: US 2012/0172727 A1
(43) Pub. Date: Jul. 5, 2012

(54) IMAGING SYSTEM

(75) Inventors: Roger Hastings, Maple Grove, MN (US); Kevin D. Edmunds, Ham Lake, MN (US); Tat-Jin Teo, Sunnyvale, CA (US); Michael J. Pikus, Golden Valley, MN (US); Leonard B. Richardson, Brooklyn Park, MN (US)

(73) Assignee: BOSTON SCIENTIFIC SCIMED, INC., Maple Grove, MN (US)

(21) Appl. No.: 13/316,839

(22) Filed: Dec. 12, 2011

Related U.S. Application Data
(60) Provisional application No. 61/469,299, filed on Mar. 30, 2011.
(60) Provisional application No. 61/428,567, filed on Dec. 30, 2010.

(51) Int. Cl. A61B 8/12

(52) U.S. Cl. 600/463

(57) ABSTRACT

Techniques are described that allow intravascular ultrasound (“IVUS”) imaging of patient tissue, e.g., a blood vessel wall, to be performed at one or more angles selected by a clinician, for example. In one example, a method includes receiving user input, via interaction with a user interface, that defines a range of angles through which a scan will be performed, determining, based on the received user input, at least one current value to be applied to at least one lead of a stator of a motor, controlling application of the at least one current to the at least one lead of the stator in order to rotate a rotor of the motor through the range of angles, and through the range of angles, receiving and processing electrical signals from at least one transducer to form at least one image.

This application claims the benefit of U.S. Provisional Application No. 61/428,567, entitled, “IMAGING SYSTEM,” by Roger Hastings, Kevin D. Edmunds, and Tat-Jin Teo, and filed on Dec. 30, 2010; and U.S. Provisional Application No. 61/469,299, entitled, “IMAGING SYSTEM,” by Roger Hastings, Kevin Edmunds, Tat-Jin Teo, Michael J. Pikus, and Leonard B. Richardson, and filed on Mar. 30, 2011, the entire contents of each being incorporated herein by reference.

### TECHNICAL FIELD

This disclosure relates to medical devices and, more particularly to intravascular ultrasound imaging devices.

### BACKGROUND

Intravascular ultrasound (“IVUS”) imaging systems provide visual indicia to a practitioner when diagnosing and treating various diseases and disorders. For example, IVUS imaging systems have been used to diagnose blocked blood vessels and to provide information to a practitioner in selecting and placing stents and other devices to restore or increase blood flow to a vessel. IVUS imaging systems have also been used to diagnose plaque build-up in the blood vessels and other intravascular obstructions. IVUS imaging systems can also be used to monitor one or more heart chambers. IVUS imaging systems are often used to visualize various portions of the vascular system that may be difficult to visualize using other imaging techniques, such as angiography, where movement caused by a beating heart or obstruction by one or more structures such as blood vessels can impair the quality of the image retrieved.

An IVUS imaging system can include a control unit, a catheter, and one or more transducers disposed in the catheter. The catheter is configured and arranged for percutaneous insertion into a patient and can be positioned in a lumen or cavity at or near a region to be imaged, such as a blood vessel wall. Electrical pulses generated by the control unit are delivered to the transducer(s) and transformed into acoustic pulses that are transmitted through the blood vessel wall or other patient tissue. Reflected pulses of the transmitted acoustic pulses are absorbed by the transducer(s) and transformed into electrical signals that are converted to an image visible by the practitioner.

### SUMMARY

In general, this disclosure describes techniques for intravascular imaging. In particular, this disclosure describes techniques that allow intravascular ultrasound (“IVUS”) imaging of patient tissue, e.g., a blood vessel wall, to be performed at one or more angles selected by a clinician, for example. Using various techniques of this disclosure, an IVUS imaging system may scan back and forth over the angular portion selected by the clinician in order to obtain a high resolution image of only the selected region.

In one example, the disclosure is directed to an imaging assembly for an intravascular ultrasound system, the imaging assembly comprising a catheter having a distal end and a proximal end, the catheter defining a catheter lumen extending from the proximal end to the distal end, the catheter configured and arranged for insertion into the vasculature of a patient, an imaging core having a distal end and a proximal end, wherein the imaging core is disposed in the distal end of the catheter lumen, wherein the imaging core defines a guidewire lumen that extends from the proximal end of the imaging core to the distal end of the imaging core, the imaging core comprising at least one transducer configured to transduce applied electrical signals to acoustic signals and also to transduce received echo signals to electrical signals, a transformer disposed in the distal end of the imaging core and about the guidewire lumen, the transformer comprising a rotating component and a stationary component, wherein the rotating component and the stationary component are spaced apart from one another, and wherein the rotating component is coupled to the at least one transducer and is configured to rotate with the at least one transducer, and a magnet disposed about the guidewire lumen, the magnet configured to be driven to rotate by a magnetic field, wherein the magnet is mechanically coupled to the at least one transducer. The imaging assembly further comprises at least one conductor electrically coupled to the stationary component of the transformer and extending to the proximal end of the catheter.

In another example, the disclosure is directed to an imaging assembly for an intravascular ultrasound system, the imaging assembly comprising a catheter having a distal end and a proximal end, the catheter defining a catheter lumen extending from the proximal end to the distal end, the catheter configured and arranged for insertion into the vasculature of a patient, an imaging core having a distal end and a proximal end, wherein the imaging core is disposed in the distal end of the catheter lumen, wherein the imaging core defines a guidewire lumen that extends from the proximal end of the imaging core to the distal end of the imaging core. The imaging core comprises at least one transducer configured to transduce applied electrical signals to acoustic signals and also to transduce received echo signals to electrical signals, a magnet disposed about the guidewire lumen, the magnet configured to be driven to rotate by a magnetic field, and a reflective surface configured to rotate with the magnet, reflect the acoustic signals from the at least one transducer into adjacent tissue, and reflect echo signals from the tissue back to the at least one transducer. The assembly further comprises at least one conductor electrically coupled to the at least one transducer and extending to the proximal end of the catheter.

In another example, the disclosure is directed to an intravascular ultrasound imaging system comprising an imaging assembly as described above in paragraphs [0005] and [0006], a user interface, and a control unit coupled to the imaging core. The control unit comprises a pulse generator electrically coupled to the at least one transducer via the at least one conductor, the pulse generator configured to generate electric signals that are applied to the at least one transducer during a scan, and a processor electrically coupled to the at least one transducer via the at least one conductor. The processor is configured to receive user input, via interaction with the user interface, that defines a range of angles through which the scan will be performed, determine, based on the received user input, at least one current value to be applied to at least one lead of a stator, control application of the at least one current to the at least one lead of the stator in order to rotate the magnet through the range of angles, and through the range of angles, receive and process electrical signals from the at least one transducer to form at least one image.

In another example, the disclosure is directed to a method for imaging a patient using an intravascular ultrasound imaging system, the method comprising receiving user input, via interaction with a user interface, that defines a range of angles through which a scan will be performed, determining, based on the received user input, at least one current value to be applied to at least one lead of a stator of a motor, controlling application of the at least one current to the at least one lead of the stator in order to rotate a rotor of the motor through the range of angles, and through the range of angles, receiving and processing electrical signals from at least one transducer to form at least one image.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 is a schematic view of one example catheter of an intravascular ultrasound imaging system, in accordance with this disclosure.

FIG. 2 is a block diagram illustrating an example control unit that may be used to implement various techniques of this disclosure.

FIG. 3 is a schematic view of one example of an imaging core that may used to implement various techniques of this disclosure.

FIG. 4 is a conceptual diagram illustrating current flow in a three-phase motor.

FIGS. 5A-5F are conceptual diagrams illustrating an ultrasound beam sweeping an arc across a vessel, in accordance with certain techniques of this disclosure.

FIG. 6 is a conceptual diagram illustrating an example catheter system monitoring blood flow in the heart of a patient, in accordance with certain techniques of this disclosure.

FIG. 7A is a schematic view of another example of an imaging core that may be used to implement various techniques of this disclosure.

FIGS. 7B and 7C are schematic longitudinal cross-sectional views of the example imaging core of FIG. 7A.

FIG. 8 is a flow diagram illustrating an example method for imaging patient tissue, in accordance with the disclosure.

### DETAILED DESCRIPTION

In general, this disclosure describes techniques that allow intravascular ultrasound (“IVUS”) imaging of patient tissue, e.g., a blood vessel wall, to be performed at one or more angles selected by a clinician, for example. Using various techniques of this disclosure, an IVUS imaging system may scan back and forth over the angular portion selected by the clinician in order to obtain a high resolution image of only the selected region. As described in more detail below, this disclosure describes how a magnetic field is generated that directs a reflective surface or transducer to any selected angle relative to fixed stator windings of a motor.

In an imaging application, an arc along the circumference of a blood vessel cross section can be selectively viewed by sweeping the mirror or transducer through angles that define the arc. In some examples, the arc is swept out at a fixed angular rate with deceleration and direction reversal occurring at the ends of the arc. In one example implementation that utilizes a stepper motor, the motor stops and dwells long enough to ping the transducer and receive the echo at multiple points along the arc. The time required to sweep out the arc is approximately equal to the arc's fraction of 360°. The number of pixels generated in the arc region in a given time (frame rate) is equal to the frame rate during normal rotational imaging divided by this fraction. For example, a 36° arc can be imaged at a frame rate that is ten times the rotational imaging frame rate.

The ability to direct ultrasound energy in any direction allows creative imaging schemes. For example, an increased frame rate can be obtained by sweeping an arc multiple times or by a single sweep that takes smaller angular steps between ultrasound bursts. When multiple sweeps are used, the imaging angles or angles at which ultrasound bursts are fired may be slightly different on each sweep. The sweep algorithm may use incremented steps or randomly chosen steps.

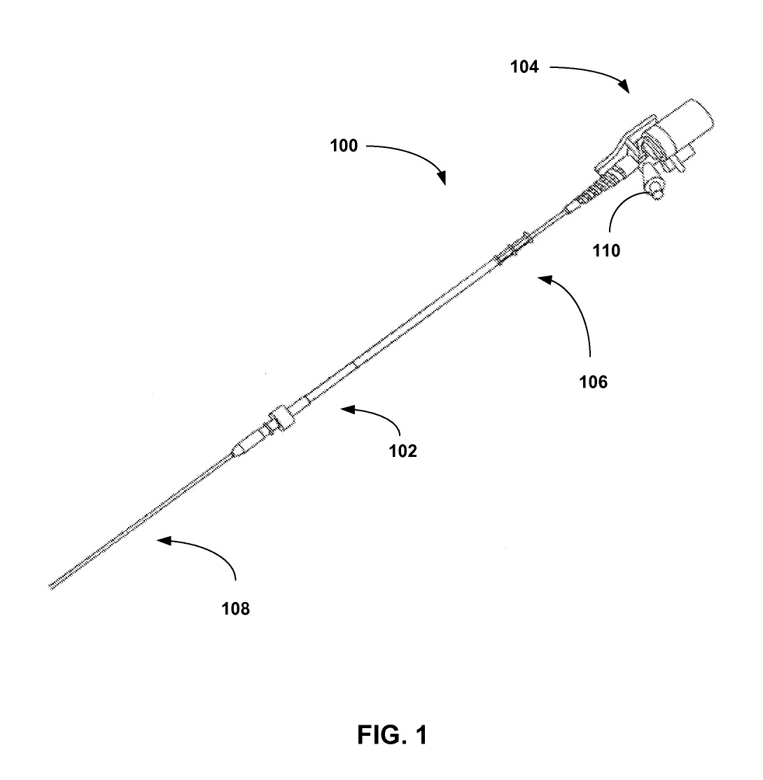

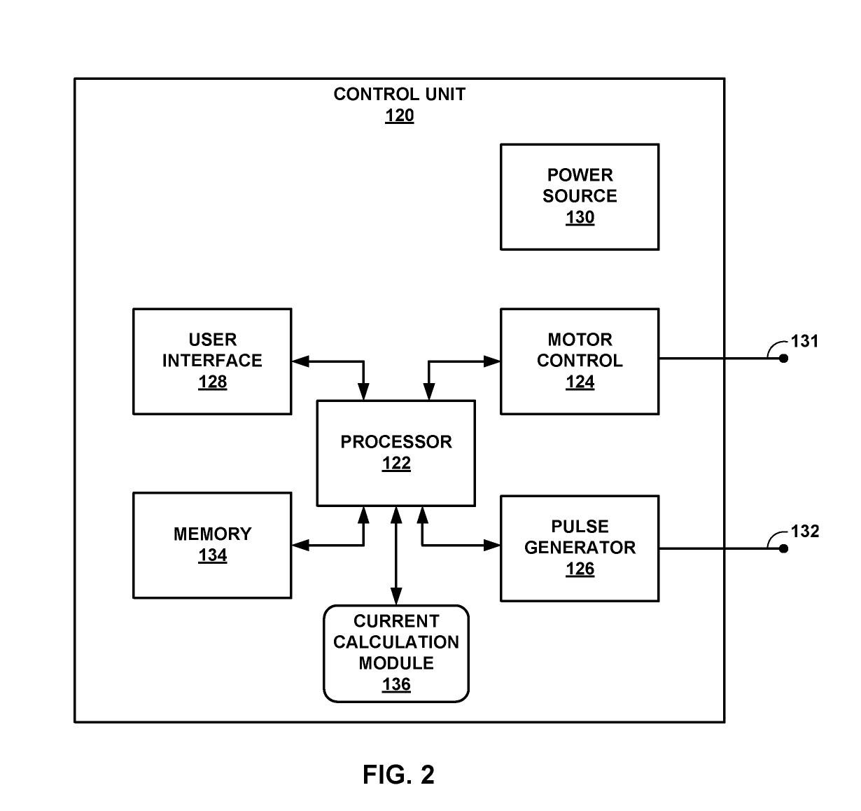

FIG. 1 is a schematic view of one example catheter of an intravascular ultrasound imaging system, in accordance with this disclosure. As seen in FIG. 1, a catheter, shown generally at 100 includes elongated member 102 and hub 104. Elongated member 102 includes proximal end 106 and distal end 108. Proximal end 106 of elongated member 102 is coupled to hub 104, and distal end 108 of elongated member 102 is configured and arranged for percutaneous insertion into a patient. In at least some example implementations, catheter 100 defines one or more flush ports, such as flush port 110. In one example, flush port 110 is defined in hub 104. In some examples, hub 104 is configured and arranged to couple to a control unit (shown in FIG. 2). In some example configurations, elongated member 102 and hub 104 are formed as a unitary body. In other examples, elongated member 102 and catheter hub 104 are formed separately and subsequently assembled together.

FIG. 2 is a block diagram illustrating an example control unit that may be used to implement various techniques of this disclosure. In the example configuration depicted in FIG. 2, control unit 120 includes processor 122 that controls motor control unit 124, pulse generator 126, and user interface 128. In some examples, electric signals, e.g., pulses, transmitted from one or more transducers are received as inputs by processor 122 for processing. In one example, the processed electric signals from the transducer(s) are displayed as one or more images on a display of user interface 128.

Processor 122 can also be used to control the functionality of one or more of the other components of the control unit 120. In one example, processor 122 is used to control at least one of the frequency or duration of the electrical signals transmitted from pulse generator 126, a rotation rate and a range of orientation angles of the imaging core by motor control unit 124, or one or more properties of one or more images formed on a display.

Processor 122 can include any one or more of a controller, a microprocessor, an application specific integrated circuit (ASIC), a digital signal processor (DSP), a field-programmable gate array (FPGA), or equivalent discrete or integrated logic circuitry. The functions attributed to processor 122 in this disclosure may be embodied as hardware, software, firmware, as well as combinations of hardware, software, and firmware.

Control unit 120 further includes power source 130. Power source 130 delivers operating power to the components of control unit 120. In one example, power source 130 includes a battery and power generation circuitry to generate the operating power.

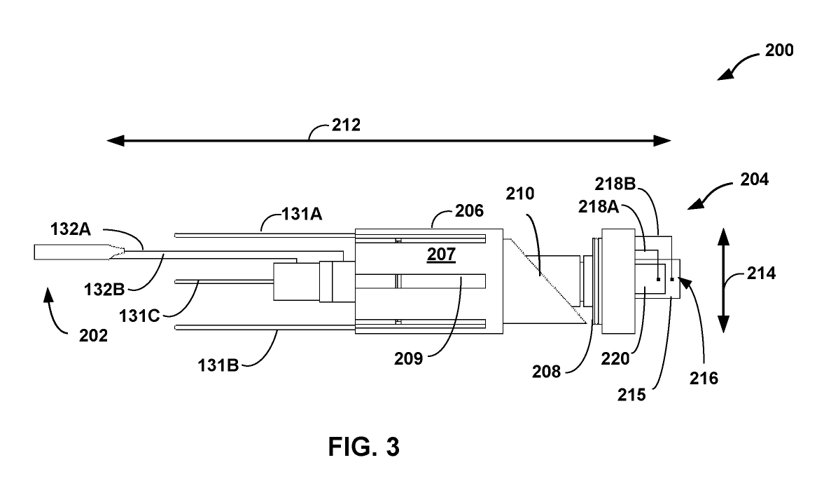

In addition, control unit 120 includes motor control unit 124. Motor control unit 124 supplies one or more current outputs to a motor (e.g., motor 206 in FIG. 3) in the imaging core of catheter 100 via one or more leads 131. As described in more detail below, current calculation module 136 determines a current to supply to the motor, and processor 122 controls motor control unit 124 to supply the determined current, e.g., three-phase direct current (DC), via lead(s) 131 in order to generate a magnetic field that directs a reflective surface or transducer to any selected angle relative to fixed stator windings of the motor.

Pulse generator 126 generates electric signals, e.g., pulses, that are applied via one or more leads 132, e.g., coaxial cable, to one or more transducers (e.g., transducer 208 of FIG. 3) disposed in catheter 100. User interface 128 includes a display, e.g., a touch screen display or another display, and in some examples, includes a keyboard, and a peripheral pointing device, e.g., a mouse, that allows a user, e.g., clinician, to provide input to control unit 120.

Control unit 120 further includes memory 134 and current calculation module 136. Memory 134 may include computer-readable instructions that, when executed by processor 122, cause processor 122 to perform various functions ascribed to control unit 120, processor 122, and current calculation module 136. The computer-readable instructions may be encoded within memory 134. Memory 134 may comprise computer-readable storage media such as a random access memory (RAM), read-only memory (ROM), non-volatile RAM (NVRAM), electrically-erasable programmable ROM (EEPROM), flash memory, or any other volatile, non-volatile, magnetic, optical, or electrical media. In one example, current calculation module 136 is encoded as instructions in memory 134 that are executed by processor 122. Using various techniques of this disclosure, a processor, e.g., processor 122, determines, based on user input defining a range of angles through which a scan will be performed, one or more current values to be applied to one or more leads of a stator of a micro-motor located in the imaging core of catheter 100, as described in more detail below.

FIG. 3 is a schematic view of one example of an imaging core that may used to implement various techniques of this disclosure. The imaging core, shown generally at 200, has proximal end 202 and distal end 204. Imaging core 200 includes motor 206, e.g., stepper motor, DC brushless motor, and one or more stationary transducers 208 configured and arranged for transducing applied electrical signals received from pulse generator 126 (FIG. 2) via leads 132A, 132B (collectively “leads 132”) to acoustic signals and also for transducing received echo signals to electrical signals.

In at least one example configuration, motor 206 is a micro-motor. Motor 206 includes stator 207 and rotatable magnet 209 (substantially hidden in FIG. 3 beneath stator 207). In some examples, motor 206 is positioned proximal to transducer(s) 208, as seen in FIG. 3. In other example implementations, motor 206 is positioned distal to transducer(s) 208. As seen in FIG. 3, motor 206 is coaxially aligned with transducer(s) 208. However, in other examples, motor 206 does not share a common axis with transducer(s) 208.

Control unit 120 is electrically connected to motor 206 via leads, e.g., three-phase leads 131A-131C (referred to herein as “leads 131”). In at least one example configuration, leads 131 and leads 132, e.g., shielded electrical cables such as coaxial cable, twisted pair cable, and the like, extend along at least a portion of the longitudinal length of the catheter 100.

Imaging core 200 further includes reflective surface 210, e.g., a mirror. Reflective surface 210 is configured to rotate with magnet 209 via a drive shaft (not shown in FIG. 3) disposed about stationary center tube 215. Reflective surface 210 reflects ultrasound energy from stationary transducer 208 to adjacent tissue of a patient and reflects echo signals from the tissue back to stationary transducer 208. Reflective surface 210 can be a reflective surface of a magnet (not shown) or, in some examples, a reflective surface either disposed on or coupled to the magnet. As seen in FIG. 3, in some example configurations, reflective surface 210 is tilted at an angle that is not parallel with either a longitudinal axis 212 of imaging core 200 or diameter 214 of imaging core 200.

In some example implementations, reflective surface 210 is tilted at an angle so that acoustic signals output from transducer(s) 208, e.g., pulses of ultrasound energy, are reflected in a direction that is not parallel to longitudinal axis 212 of imaging core 200. In at least one example, reflective surface 210 is tilted at an angle so that acoustic signals output from transducers 208, e.g., pulses of ultrasound energy, are reflected toward patient tissue in a direction that is roughly perpendicular to the longitudinal length 212 of imaging core 212.

Reflective surface 210 is tilted at an angle so that at least some of the echo signals received from patient tissue (in response to the acoustic signals output from transducer(s) 208) are reflected to transducers 208. The echo signals are transduced into electric signals and transmitted to processor 122 for processing in order to produce an image. In at least some examples, reflective surface 210 is tilted at an angle so that at least some of the echo signals from patient tissue are reflected to a direction that is parallel to longitudinal axis 212 of imaging core 200.

In one example configuration, every other strip in stator 207 is driven, while intervening strips are for structure, and are not electrically active. Three phase current is applied to three stator leads, causing magnet 209 and reflective surface 210 to rotate to the specified angle(s). Distal transducer 208 launches ultrasound pulses that reflect from reflective surface 210 into adjacent tissues.

As mentioned above, imaging core 200 further includes stationary center tube 215, which defines a guidewire lumen, shown generally at 216. In the example shown in FIG. 3, center tube 215 extends from proximal end 202 of imaging core 200 to distal end 204 of imaging core 200. As seen in FIG. 3, motor 206, transducer 208, and reflective surface 210 are disposed about guidewire lumen 216, thereby allowing guidewire lumen 216 to extend completely through the imaging core. Transducer 208 is electrically connected to leads 132, e.g., a coaxial cable, via leads 218A and 218B. In particular, lead 218A is connected to conductive film 220, which is adhered to center tube 215, and lead 218B is connected to center tube 215. In this manner, the example configuration depicted in FIG. 3 uses conductive film 220 as a first conductor and center tube 215 as a second conductor.

Additional details regarding IVUS imaging systems may be found, for example, in the following references: U.S. Pat. Nos. 6,945,938 and 7,306,561; U.S. Patent Application Publication Nos. 2006/0100522; 2006/0253028; 2007/0016054; 2007/0003811; 2010/0249599; 2010/0249603; and 2010/0249604; and U.S. application Ser. Nos. 12/565,632 and 12/566,390, each of which is incorporated by reference herein in its entirety.

Using various techniques of this disclosure, an IVUS imaging system may scan back and forth over an angular portion selected or defined by a clinician in order to obtain a high resolution image of the selected or defined region. In particular, this disclosure describes certain techniques that generate a magnetic field that directs a reflective surface, e.g., reflective surface 210 of FIG. 3, or a transducer (shown and described in more detail below) to any selected angle relative to the fixed stator windings. For example, by directing the reflective surface or transducer to a selected angle, a practitioner, e.g., a clinician, physician, or other medical professional, may select only viewing angles that contain plaque. Or, as another example, dwelling at a fixed angle selected by the clinician can be used to obtain a Doppler image of blood flow in the direction of the selected angle.

In accordance with certain techniques of this disclosure, control unit 120 and, in particular, processor 122, receives user input from a clinician that defines an angle or range of angles through which the clinician would like to perform a scan. Based on the received user input, processor 122 then determines, via current calculation module 136, one or more current values to be applied to one or more leads 131 of a stator of motor 206. Via motor control unit 124, processor 122 controls application of the determined current(s) to the lead(s) 131 of the stator in order to rotate a rotor of motor 206 to the selected angle or through the selected range of angles. At the selected angle or through the selected range of angles, processor 122 receives and processes electrical signals from one or more transducer(s), e.g., transducer 208, to form one or more images.

As indicated above, control unit 120 and, in particular, processor 122, receives user input from a clinician that defines an angle or range of angles through which the clinician would like to perform a scan. In some examples, user interface 128 can include a touch screen for receiving user input. In such an example, the clinician can use a stylus, finger, or other pointing device to outline on an anatomical representation of the region of interest displayed on the touch screen, e.g., a blood vessel wall, a range of angles through which the clinician would like to perform a scan. In another example, the clinician can use a stylus, finger, or other pointing device to define the range of angles by touching a starting point and an ending point on an anatomical representation of the region of interest displayed on the touch screen. In example implementations that do not use a touch screen, the clinician can use peripheral pointing device, e.g., a mouse, trackball, or the like, to outline a range of angles or specify starting and ending points.

In one example implementation, user interface 128 may include a keyboard by which a clinician may enter starting and ending angles. Or, a clinician may use pull down menus to select particular starting and ending angles. In other example implementations, user interface 128 allows a clinician to specify particular quadrants of interest, or other ranges of angles, rather than selecting particular starting and ending angles.

In example configurations in which motor 206 is a stepper motor, a clinician may specify, via user interface 122, a number of steps for the stepper motor to advance. For example, if each step advances stepper motor 206 by 3.6° and if the clinician would like to scan a range of 36°, then ten steps are needed. As such, the clinician may use user interface 128 to specify ten steps. Of course, this is only one specific example; stepper motor 206 may be advanced by steps greater or less than 3.6° and ranges greater or less than 36° can be scanned.

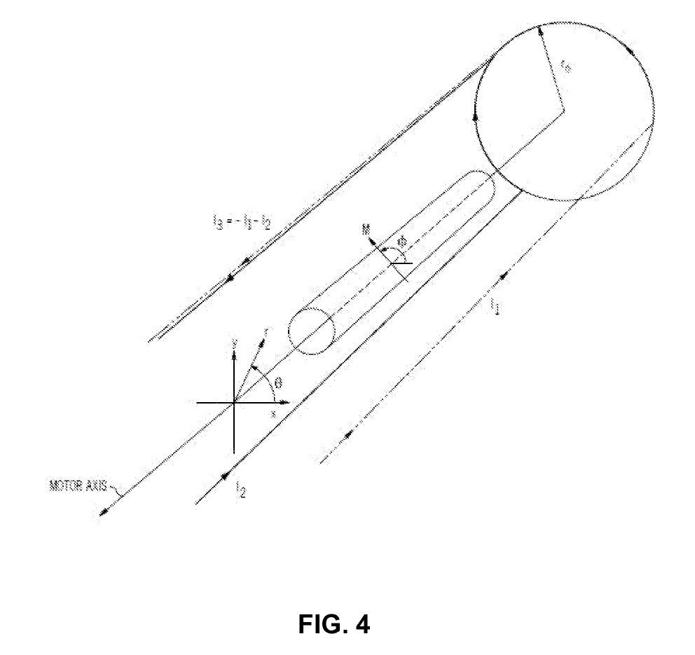

As indicated above, based on the received user input, processor 122 determines, via current calculation module 136, one or more current values to be applied to one or more leads 131 of a stator of motor 206. In one example implementation of the techniques of this disclosure, motor 206 is a three-phase DC motor. Without wishing to be bound by any theory, the principle of operation for determining the current values to be applied to the stator of a motor, e.g., three-phase DC motor, in order to generate a magnetic field that directs a reflective surface or transducer to any selected angle relative to fixed stator windings of the motor, are described in detail below with respect to FIG. 4.

FIG. 4 is a conceptual diagram illustrating current flow in a three-phase motor. In particular, FIG. 4 depicts a three phase winding of a three-phase motor driven with current I₁, I₂ and a common return leg, relative to a central axis of the motor (“motor axis”). A magnetic field may be directed along any unit vector, r, by selecting the currents such that:

I₁=I₀ sin(θ),

I₂=I₀ sin(θ+120°), and

I₃=−I₁−I₂=I₀ sin(θ+240°).

The two driven legs in the three phase motor, namely I₁ and I₂, are located at 0° and −120° relative to the central axes of the motor. The common return current I₃ automatically sums to the third phase at −240°. The magnetic field vector generated by the line currents is located at angle θ and is directed radially outward.

The principle of operation of a three phase winding is based on the following trigonometry identity, which may be verified by expanding the terms on the left:

sin(θ)+sin(θ+120°)+sin(θ+240°)=0 (1)

The identity of Eq. (1) is valid for all angles θ.

The two driven current legs and the passive return current leg in the three phase motor shown in FIG. 4 are geometrically located at 0°, −120°, and −240° relative to the coordinate system shown in the figure, and carry currents proportional to the three terms on the left of Eq.(1),

I₁=I₀ sin(θ) (2)

I₂=I₀ sin(θ+120°) (3)

I₃=−I₁−I₂=I₀ sin(θ+240°), (4)

where I₁ is the first phase driven current in amps, I₂ is the second phase driven current in amps, and I₃, which equals −I₁−I₂, is the third phase passive return current in amps.

The torque on a motor magnet of a three-phase motor is given by the following equation:

τ=m×H (5)

where τ is the torque on the magnet in Newton-meters (Nt-m), m is the magnet magnetic moment in Tesla-m³, H is the magnetic field from the three windings in Amp/m, and where bold face type in Eq. (5) denotes vector quantities. It should be noted that the “x” in Eq. (5) denotes the vector cross product.

Neglecting any magnetic fields from the winding ends, the fields from the three line currents in the figure form circles around each line winding, and along the magnet axis are given by the following equations:

H₁=[I₀ sin(θ)/(2π₀)]j (6)

H₂=[I₀ sin(θ+120°)/(2πr₀)](sin(120°)i+cos(120°)j) (7)

H₃=[I₀ sin(θ+240°)/(2πr₀)](sin(240°)i+cos(240°)j) (8)

where i, j, and k are unit vectors along the x, y, and z axis respectively, I₀ is the amplitude of the current in each winding, and r₀ is the separation between the motor axis and the windings (e.g., radius of the stator).

The net magnetic field is the sum of H₁, H₂, and H₃ in Eqs. (6)-(8) above, which equals:

H=[3I₀/(4πr₀)]r (9)

where r=cos(θ) i+sin(θ) j=radial unit vector at angle θ.

Finally, the torque on the magnet can be computed from Eq. (5). The magnetic moment in Eq. (5) is given by the following equation:

m=MV(cos(φ)i+sin(φ)j) (10)

where M is the magnet magnetization in Tesla, V=magnet volume in m³, and φ=angle between the x axis and the magnetization vector.

Because both the torque and magnetic field lie in the x-y plane, the cross product in Eq. (5), computed from Eqs. (9) and (10), is given by the following equation:

τ=[3MVI₀/(4πr₀)] sin(θ−φ)k (11)

Using Eq. (11) in the equation of motion for the magnet shows that a steady state solution is the following:

φ=θ (12)

That is, the magnetization vector of the magnet is aligned with the magnetic field direction. U.S. application Ser. No. 12/566,390, incorporated herein by reference in its entirety, describes the acceleration of the magnet when magnetic torque is applied, and shows that the magnet can reach steady state very rapidly. Viscous drag between the magnet bearing surfaces creates a small lag between the orientation of the magnetization and the applied field.

In rotational IVUS, the magnetic field is rotated at a uniform rate, and the magnet angle is given by the following equation:

φ=2πf*t (13)

where f equals the magnet rotation rate (nominally 30 Hz for IVUS), and t=time in seconds. In general,

φ=θ(t) (14)

where θ(t) is a user specified function of time.

A given angle is achieved in steady state when the three phase stator windings are energized with the currents given by Eqs. (2)-(4). For example, the magnet angle may be swept back and forth over an arc of interest, with deceleration and motion reversal occurring in a short time at the ends of the arc. Movement of the magnet in steps, with a dwell time at each step in which the magnet is held in a fixed orientation, is described in detail in U.S. application Ser. No. 12/566,390. Although rotational stepper motor action is discussed in U.S. application Ser. No. 12/566,390, the net motion can describe any user specified set of viewing angles versus time. As one example implementation, steps can be taken over an arc, with no angular positions repeated in successive sweeps over the arc. Such an approach can provide more distinct pixels in a given arc of tissue.

Using the techniques of this disclosure, a clinician enters a range of angles or a specific angle, via interaction with a user interface, e.g., user interface 128, which defines a range of angles or specific angle through which a scan will be performed. Control unit 120 and, in particular, current calculation module 122 under the control of processor 122, determines, based on the received user input, at least one current value to be applied to at least one lead of a stator of a motor, e.g., motor 206, using one or more of equations (1)-(14) described above. After the current values have been determined, processor 122 controls application of the current to the at least one lead of the stator, via motor control unit 124, in order to rotate a rotor of the motor through the range of angles selected by the clinician. Through the range of angles selected by the clinician, processor 122 receives and processes electrical signals from transducer(s) 208 to form one or more images, e.g., ultrasound images.

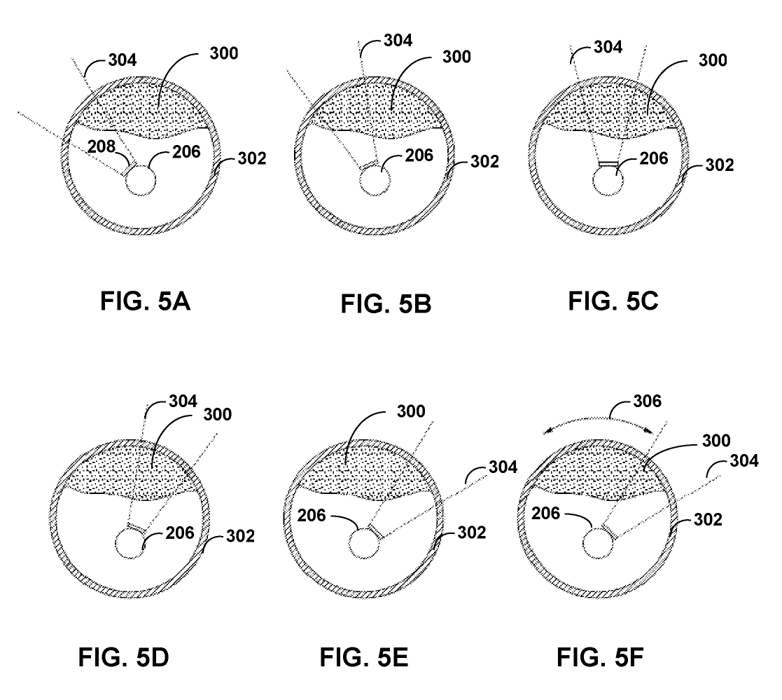

FIGS. 5A-5F are conceptual diagrams illustrating an ultrasound beam sweeping an arc across a vessel, in accordance with certain techniques of this disclosure. In particular, FIGS. 5A-5F depicts motor 206 rotating transducer 208 through a range of angles in order to scan plaque 300 attached to artery wall 302 using ultrasound beam 304 (generated by transducer 208). Generally speaking, in one example implementation, a transducer, e.g., transducer 208, is first rotated to obtain a 360° view of artery wall 302. The clinician determines that she would like a more detailed look at the region of artery wall 302 that contains plaque 300. The clinician sets control unit 120 to select only the viewing angles that contain plaque 300. The micro-motor then scans transducer 208 back and forth across the span of selected angles to produce a relatively high resolution image of the selected plaque.

FIG. 5A depicts ultrasound beam 304 oriented at a first, or starting, angle and scanning plaque 300. FIG. 5E depicts ultrasound beam 304 oriented at a second, or ending, angle and scanning plaque 300. FIGS. 5B-5D depict ultrasound beam 304 oriented at various intervening angles between the starting and ending angles. As described above, a clinician may specify, via user interface 128, the starting angle and ending angle, for example, through which motor 206 will rotate and thus ultrasound beam 304 will scan. FIG. 5F depicts that, in some example implementations, ultrasound beam 304 can scan plaque 300 back and forth, as indicated by arrow 306.

Using various techniques described above, motor 206, e.g., a micro-motor, can be rapidly stopped and adjusted to precise angular positions. In addition, the clinician can select angles relative to the full 360° image of the artery wall, as in the example of FIGS. 5A-5F. In other example implementations, one or more magnetic field sensors outside of the patient can sense the magnetic field of the micro-motor magnet and determine its absolute orientation in a fixed reference system. This allows the IVUS image to be registered to other images such as a pre-operative computed tomography (CT) scan or a real time fluoroscope image

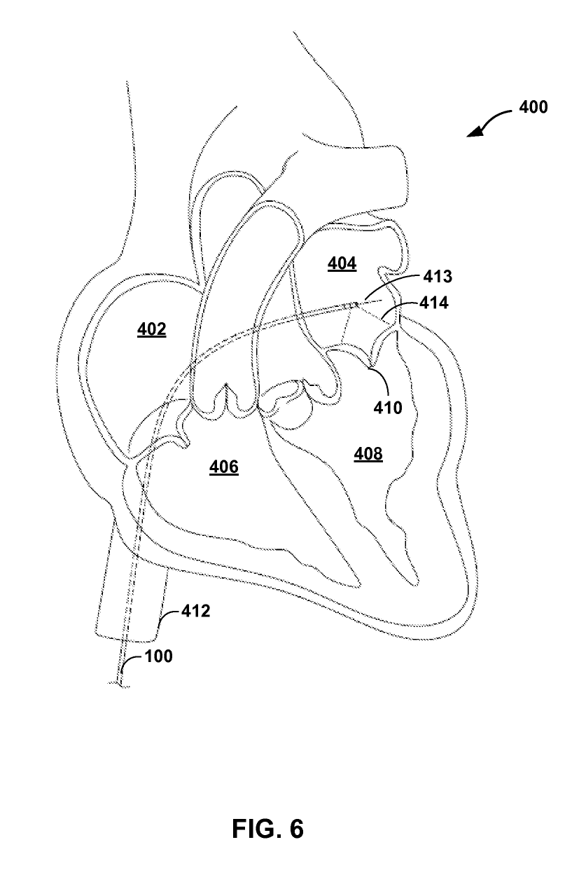

FIG. 6 is a conceptual diagram illustrating an example application of a catheter system that monitors blood flow in the heart of a patient, in accordance with certain techniques of this disclosure. In particular, FIG. 6 depicts a clinical application of the ability to stop a transducer of an imaging system such that the transducer is pointing in a selected direction. FIG. 6 depicts a heart, shown generally at 400, having right atrium 402, left atrium 404, right ventricle 406, and left ventricle 408. Mitral valve 410 lies between left atrium 404 and left ventricle 408. In the specific example shown in FIG. 6, micro-motor driven IVUS catheter 100 has been advanced through inferior vena cava 412 along optional guidewire 413 across the atrial septum (not shown) and into left atrium 404 to treat atrial fibrillation or to repair the mitral valve, for example. It should be noted that in other example implementations, catheter 100 may be advanced without the use of a guidewire. Micro-motor driven IVUS catheter 100 is advanced into left atrium 404 in order to assess blood flow through mitral valve 410, for example, to determine the degree of mitral valve regurgitation.

Using various techniques of this disclosure, processor 122 (FIG. 2) controls transducer 208 to rotate or sweep through angles that point toward the mitral valve in order to determine its cross sectional area for blood flow. Processor 122 (FIG. 2) controls the rotation of motor 206 (FIG. 3) such that transducer 208 (FIG. 3) stops and points directly at mitral valve 410 with an ultrasound beam 414. A transducer, e.g., transducer 208 (FIG. 3), via pulse generator 126, directs ultrasound beam 414 into the blood flow (not shown) and processor 122 measures the frequency of echos received by transducer 208 (FIG. 3).

In addition, processor 122 determines the Doppler shift, or difference in frequency between the outgoing and reflected beams. The Doppler shift has a known relationship to blood flow velocity. The product of the area of mitral valve 410 and the Doppler flow velocity determines volumetric blood flow rate (milliliters/minute). When mitral valve 410 is closed, regurgitating blood flows toward transducer 208, thereby reversing the sign of the Doppler shift. Processor 122 estimates the area of a leak when mitral valve 410 is closed, and then determines the ratio of regurgitated to normal blood flow.

To summarize the example application depicted in FIG. 6, a micro-motor driven IVUS catheter is advanced across the atrial septum to determine blood flow through the mitral valve. An image of the valve is first acquired to determine its area. A transducer is pointed directly into the blood flow and the frequency shift of the reflected beam (Doppler shift) is measured to compute blood flow velocity. The product of the valve area and blood flow velocity determines blood flow rate.

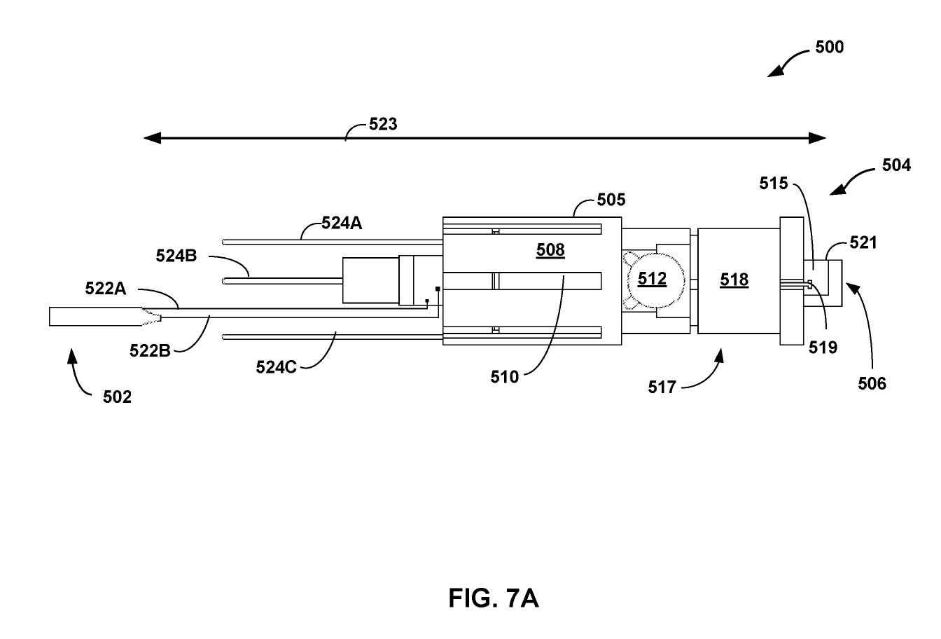

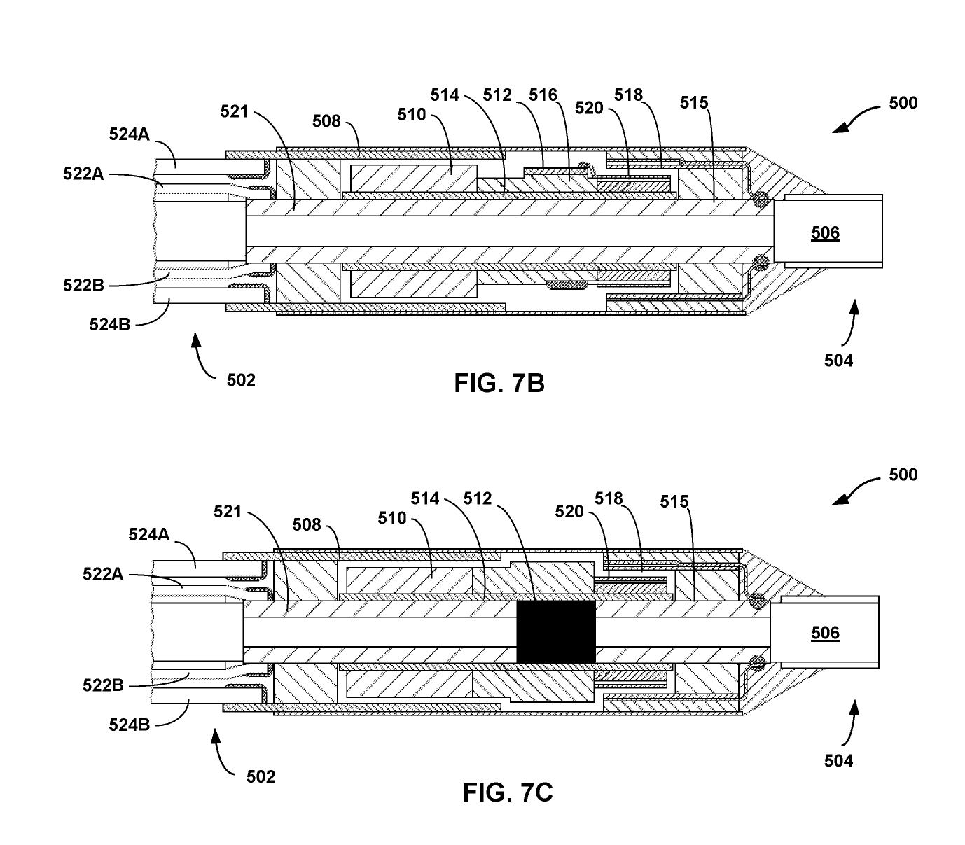

As indicated above with respect to FIG. 3, an imaging core, e.g., imaging core 200, can include a reflective surface 210 configured to reflect ultrasound pulses from a transducer, e.g., transducer 208, toward patient tissue and receive echo signals from the patient tissue (in response to the acoustic signals output from transducer(s) 208). In accordance with certain techniques of this disclosure, however, the imaging core can be configured to include a distal transformer and side-looking transducer, instead of a reflective surface, as described in detail below with respect to FIGS. 7A-7C.

FIG. 7A is a schematic view of another example of an imaging core that may be used to implement various techniques of this disclosure. In particular, FIG. 7A depicts one example of an imaging core of an IVUS catheter system having a distal transformer and side-looking transducer that can scan back and forth over an angular portion selected by a clinician in order to obtain a high resolution image of only the selected region.

Generally speaking, in one example implementation, an IVUS control unit transmits voltage pulses down a transducer coaxial cable and into a primary winding, or coil, of a distal transformer located near the catheter tip. The pulse is inductively coupled to a rotating transformer secondary winding, or coil, to transmit the ultrasound pulse from the transducer toward adjacent patient tissue. The pulse is reflected from the adjacent tissue and returns to the transducer where it is converted to a voltage echo, and is inductively coupled from the moving transformer secondary winding to the fixed primary winding, and back to the IVUS control unit for processing and display. The transducer can be steered to any selected or programmed angles using the techniques described above.

The imaging core, shown generally at 500, has proximal end 502 and distal end 504. Imaging core 500 includes motor 505. In at least one example configuration, motor 505 is a micro-motor. Motor 505 includes stator 508 and rotatable magnet 510 (substantially hidden in FIG. 7A beneath stator 508). Rotatable magnet 510 is configured to be driven to rotate by a magnetic field generated within stator 508 that surrounds magnet 510.

Imaging core 500 of FIG. 7A further includes rotating ultrasound transducer 512 and a distal transformer, shown generally at 517. Transducer 512 is mechanically coupled to rotatable magnet 510 by a drive shaft (shown at 514 in FIGS. 7B and 7C) that is disposed about stationary center tube 521. Distal transformer 517 includes stationary primary coil 518 and a rotating secondary coil (not shown in FIG. 7A). The rotating secondary coil is coupled to transducer 512 and is configured to provide electrical pulses to and receive electrical echo signals from transducer 512. Although transducer 512 is depicted as substantially circular in shape in FIG. 7A, transducer 512 is not limited to a substantially circular shape. Rather, in other example implementations, transducer 512 may be, for example, oval-shaped, square-shaped, rectangular-shaped (seen in the example configuration of FIG. 7C), or various other shapes not explicitly recited in this disclosure.

Primary coil lead 519 of primary coil 518 is connected to metal film interconnect 515, an electrical conductor, which is adhered to stationary center tube 521 and which carries transformer electrical signals to and from the primary coil 518, underneath the drive shaft (not shown in FIG. 7A) to proximal transducer leads 522A and 522B. Electrical pulses from metal film interconnect 515 are inductively coupled from stationary primary coil 518 of transformer 517 to rotating secondary coil 520 (FIGS. 7B and 7C) of transformer 517 to energize transducer 512. Echo electrical signals from transducer 512 are inductively coupled from rotating secondary coil 520 (FIGS. 7B and 7C) of transformer 517 to stationary primary coil 518 of transformer 517 to be received at the proximal end of the catheter through at least one electrical conductor, e.g., transducer leads 522A, 522B.

As mentioned above, imaging core 500 further includes stationary center tube 521. Center tube 521 defines a guidewire lumen, shown generally at 506, which extends from proximal end 502 to distal end 504, thereby allowing a guidewire (not shown) to extend through imaging core 500 along longitudinal axis 523.

FIGS. 7B and 7C are schematic longitudinal cross-sectional views of the example imaging core shown in FIG. 7A. In particular, FIGS. 7B and 7C depict a side view and a top view, respectively, of imaging core 500 of FIG. 7A that, in accordance with this disclosure, can be used by a micro-motor driven IVUS catheter system to adjust or rotate a side-looking ultrasound transducer so that the system may scan back and forth over an angular portion selected by the clinician in order to obtain a high resolution image of only the selected region. Imaging core 500 of FIGS. 7A-7C is configured to implement any of the techniques described above with respect to FIGS. 5A-5F and 6. For purposes of conciseness FIGS. 7B and 7C will be described together.

As seen in FIGS. 7B and 7C, imaging core 500 has proximal end 502 and distal end 504, and imaging core 500 defines guidewire lumen 506, which extends from proximal end 502 to distal end 504. As such, a guidewire (not shown) may extend through imaging core 500 via guidewire lumen 506.

In addition, imaging core 500 includes a micro-motor that includes stator 508 and a rotor shown as magnet 510. Side-looking transducer 512 is coupled to magnet 510 via at least a portion of a circumference of rotatable drive shaft 514, thereby allowing transducer 512 to rotate as magnet 510 rotates. Drive shaft 514 is a tube that rotates about center tube 521 of imaging core 500. As seen in FIGS. 7B and 7C, magnet 510 is disposed about guidewire lumen 510 and configured and arranged to be driven to rotate by a magnetic field.

Transducer 512 is configured and arranged for transducing applied electrical signals to acoustic signals and also for transducing received echo signals to electrical signals. As seen in FIG. 7B, in some example configurations, imaging core 500 includes transducer backing material 516 disposed between transducer 512 and drive shaft 514. In at least one example configuration, imaging core 500 includes metal film interconnect 515 that is adhered to stationary center tube 521 and carries transformer electrical signals to and from transducer primary coil 518, underneath drive shaft 514 to proximal transducer leads 522A and 522B.

As seen in FIGS. 7B and 7C, with the use of a micro-motor, drive shaft 514 is disposed within imaging core 500. As such, non-uniform rotational distortion (NURD) is reduced or eliminated from images. NURD arises when a rotating drive shaft runs the length of the catheter, passing through the twists and turns of a blood vessel system.

Ultrasound pulses transmitted by transducer 512 are coupled through transformer 517 (FIG. 7A) that includes primary windings 518 and secondary windings 520 spaced apart from one another. In some example implementations, primary windings 518 are stationary and secondary windings 520 are configured to rotate. As shown in FIGS. 7B and 7C, the transformer with primary windings 518 and secondary windings 520 is disposed in distal end 504 of imaging core 500 about guidewire lumen 506. Secondary windings 520 are coupled to transducer 512 and are configured and arranged to rotate. In at least one example implementation, a control unit, e.g., control unit 120, transmits and receives electric signals from transducer 512 via leads 522A, 522B, or conductors, extending from primary windings 518 through metal film interconnect 515. As such, in one example, ultrasound pulses and echo signals are coupled through a fixed primary, moving secondary transformer. A processor, e.g., processor 122, determines stator currents using various techniques described above to direct the transducer to face target tissues. In some examples a control unit, e.g., control unit 120, delivers current to stator 508 via leads 524A, 524B.

Various aspects of imaging core 200 described above with respect to FIG. 3 are applicable to imaging core 500 of FIGS. 7A-7C. For example, in some examples, stator 508 comprises a three-phase winding geometry for receiving three-phase current. As another example, a sensing device that is constructed and arranged to sense an angular position of the magnet can be included in some implementations.

In this manner, certain techniques of this disclosure are directed to an imaging assembly for an intravascular ultrasound system, and an imaging system using an intravascular ultrasound imaging system. In one example configuration, the imaging assembly includes a catheter, e.g., catheter 100, an imaging core, e.g., imaging core 200, and at least one conductor, e.g., leads 132. The imaging system includes, in one example configuration, an imaging assembly, as described above, a user interface, e.g., user interface 128, and a control unit, e.g., control unit 120.

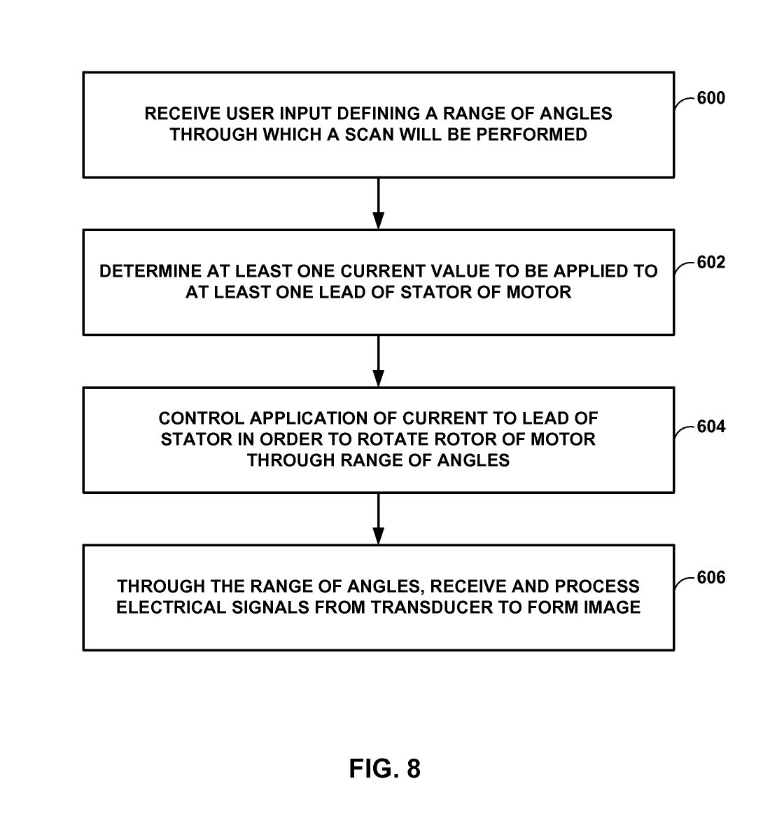

FIG. 8 is a flow diagram illustrating an example method for imaging patient tissue, in accordance with the disclosure. In FIG. 8, a processor, e.g., processor 122 of FIG. 2, receives user input from a clinician, via interaction with a user interface, e.g., user interface 128 of FIG. 2, that defines a range of angles through which a scan will be performed (600). Processor 122 then determines, based on the received user input, one or more current values, e.g., I₁ and I₂ of FIG. 4, to be applied to one or more leads of a stator of a motor (602). In some examples, the motor is part of an imaging core such as imaging core 200 of FIG. 3. In other examples, the motor is part of an imaging core such as imaging core 500 of FIGS. 7A-7C. Processor 122 controls application of the current to the lead(s) of the stator, e.g., via motor control unit 124, in order to rotate a rotor of the motor through the range of angles (604). Through the range of angles, processor 122 receives and processes electrical signals from a transducer, e.g., transducer 208 or 512, to form at least one image.

Many examples of the disclosure have been described. These and other examples are within the scope of the following claims. Various modifications may be made without departing from the scope of the claims.

1. An imaging assembly for an intravascular ultrasound system, the imaging assembly comprising:
a catheter having a distal end and a proximal end, the catheter defining a catheter lumen extending from the proximal end to the distal end, the catheter configured and arranged for insertion into the vasculature of a patient;
an imaging core having a distal end and a proximal end, wherein the imaging core is disposed in the distal end of the catheter lumen, wherein the imaging core defines a guidewire lumen that extends from the proximal end of the imaging core to the distal end of the imaging core, the imaging core comprising
at least one transducer configured to transduce applied electrical signals to acoustic signals and also to transduce received echo signals to electrical signals,
a transformer disposed in the distal end of the imaging core and about the guidewire lumen, the transformer comprising a rotating component and a stationary component, wherein the rotating component and the stationary component are spaced apart from one another, and wherein the rotating component is coupled to the at least one transducer and is configured to rotate with the at least one transducer, and
a magnet disposed about the guidewire lumen, the magnet configured to be driven to rotate by a magnetic field, wherein the magnet is mechanically coupled to the at least one transducer; and
at least one conductor electrically coupled to the stationary component of the transformer and extending to the proximal end of the catheter.

2. The imaging assembly of claim 1, wherein the magnet is engaged to a rotatable drive shaft, and wherein the at least one transducer is coupled to a portion of a circumference of the driveshaft.

3. The imaging assembly of claim 1, wherein the magnet forms a part of a stepper motor.

4. The imaging assembly of claim 1, further comprising a sensing device that is constructed and arranged to sense an angular position of the magnet.

5. The imaging assembly of claim 4, wherein the sensing device is located outside of the patient.

6. The imaging assembly of claim 1, further comprising a stator, the stator comprising a three-phase winding geometry for receiving three-phase current.

7. The imaging assembly of claim 4, wherein the stator receives the three-phase current via a control unit coupled to the imaging core, the control unit comprising:
a pulse generator electrically coupled to the at least one transducer via the at least one conductor, the pulse generator configured to generate electric signals that are applied to the at least one transducer during a scan; and
a processor electrically coupled to the at least one transducer via the at least one conductor, the processor configured to:
receive user input, via interaction with a user interface, that defines a range of angles through which the scan is performed;
determine, based on the received user input, at least one current value to be applied to at least one lead of a stator;
control application of the at least one current to the at least one lead of the stator in order to rotate the magnet through the range of angles; and
through the range of angles, receive and process electrical signals from the at least one transducer to form at least one image.

8. A method for imaging a patient using an intravascular ultrasound imaging system, the method comprising:
receiving user input, via interaction with a user interface, that defines a range of angles through which a scan will be performed;
determining, based on the received user input, at least one current value to be applied to at least one lead of a stator of a motor;
controlling application of the at least one current to the at least one lead of the stator in order to rotate a rotor of the motor through the range of angles; and
through the range of angles, receiving and processing electrical signals from at least one transducer to form at least one image.

9. An imaging assembly for an intravascular ultrasound system, the imaging assembly comprising:
a catheter having a distal end and a proximal end, the catheter defining a catheter lumen extending from the proximal end to the distal end, the catheter configured and arranged for insertion into the vasculature of a patient;
an imaging core having a distal end and a proximal end, wherein the imaging core is disposed in the distal end of the catheter lumen, wherein the imaging core defines a guidewire lumen that extends from the proximal end of the imaging core to the distal end of the imaging core, the imaging core comprising
at least one transducer configured to transduce applied electrical signals to acoustic signals and also to transduce received echo signals to electrical signals,
a magnet disposed about the guidewire lumen, the magnet configured to be driven to rotate by a magnetic field, and
a reflective surface configured to rotate with the magnet, reflect the acoustic signals from the at least one transducer into adjacent tissue, and reflect echo signals from the tissue back to the at least one transducer; and
at least one conductor electrically coupled to the at least one transducer and extending to the proximal end of the catheter.

10. An intravascular ultrasound imaging system comprising:
the imaging assembly of either of claim 1 or claim 9;
a user interface; and
a control unit coupled to the imaging core, the control unit comprising:
a pulse generator electrically coupled to the at least one transducer via the at least one conductor, the pulse generator configured to generate electric signals that are applied to the at least one transducer during a scan; and
a processor electrically coupled to the at least one transducer via the at least one conductor, the processor configured to:
receive user input, via interaction with the user interface, that defines a range of angles through which the scan will be performed;
determine, based on the received user input, at least one current value to be applied to at least one lead of a stator;
control application of the at least one current to the at least one lead of the stator in order to rotate the magnet through the range of angles; and
through the range of angles, receive and process electrical signals from the at least one transducer to form at least one image.

11. The imaging system of claim 10, wherein the user interface comprises a touch screen.

12. The imaging system of claim 11, wherein the processor receives user input outlining the range of angles through which the scan is performed.

13. The imaging system of claim 10, wherein the processor receives user input specifying a starting angle and an ending angle of the range of angles.

14. The imaging system of claim 10, wherein the magnet forms a part of a stepper motor.

15. The imaging system of claim 14, wherein the processor receives user input specifying a number of steps for the stepper motor.

16. The imaging system of claim 15, wherein the processor is further configured to determine a minimum step size for the stepper motor.
